(19) United States
(12) Patent Application Publication
(10) Pub. No.: US 2023/0347354 A1
(43) Pub. Date: Nov. 2, 2023

(54) Crucible Coupler for a Carbon Aerosol Generator

(71) Applicant: Cyclomedica Australia Pty Limited, Kingsgrove, New South Wales (AU)

(72) Inventors: Mathew Farag, Kingsgrove, New South Wales (AU); Zbigniew Bogulski, Kingsgrove, New South Wales (AU); Gabino Israel Huerta Medina, Kingsgrove, New South Wales (AU)

(73) Assignee: Cyclomedica Australia Pty Limited, Kingsgrove, New South Wales (AU)

(21) Appl. No.: 18/246,283

(22) PCT Filed: Sep. 23, 2021
(86) PCT No.: PCT/AU2021/051113

§ 371 Date: Mar. 22, 2023

(30) Foreign Application Priority Data
Sep. 24, 2020 (AU) 2020903443

(51) Int. Cl. B01L 9/00

(52) U.S. Cl. CPC B01L 9/50; B01L 2200/025

(57) ABSTRACT

A post unit for holding one end of a carbon crucible utilised in the creation of a carbon encapsulated substance such as Technetium for use in medical imaging and treatment, the post unit including: a detachable contact unit including a first cavity for engaging one end of the carbon crucible; and at least a first and second pedestal for engaging with the contact unit; the first and second pedestals being resiliently clamped together around the first contact unit for engagement of the contact unit in a predetermined position.

### RELATED APPLICATION

The present disclosure claims benefit of priority to Australian Provisional Patent Application Number: 2020903443 filed 24 Sep., 2020, the contents of which are incorporated herein by reference. In jurisdictions where incorporation by reference is not permitted, the applicant reserves the right to add any or the whole of the contents of said Application 2020903443 as an Appendix hereto, forming part of the specification.

### FIELD OF THE INVENTION

The present invention provides for systems and methods for the production of radioactive aerosols suitable for use in medical treatment, and in particular discloses a new form of dispenser arrangement and crucible holder for the production of the aerosol.

### BACKGROUND OF THE INVENTION

Any discussion of the background art throughout the specification should in no way be considered as an admission that such art is widely known or forms part of common general knowledge in the field.

The production of carbon encased radioactive isotopes such as technetium (Tc-99m) has become very important for the production of imaging media for lung imaging etc. The resultant ultra-fine suspension of carbon nano-particles is especially effective for imaging.

Examples of a system for carbon encapsulated capture production system can be found in PCT application PCT/AU2006/000239 filed 24 Feb., 2006 entitled “Improved Process for the Production of a Radioactive Aerosol”, the contents herewith are incorporated by cross reference. One form of suitable production system is the Cyclomedica TechnegasPlus Generator available from Cyclomedica Australia Pty Ltd.

FIG. 1 illustrates an example of the Cyclomedica TechnegasPlus Generator. The Technegas Generator 1 is constructed in accordance with the teaching of the aforementioned PCT application.

The loading of a crucible of the prior art device will now be explained.

Referring to both FIG. 2 and FIG. 3, there is illustrated the initial loading of a crucible of such a generator 1. The steps include the opening of a draw 2 and the placing of a crucible 5 between two conductive posts 3, 4. FIG. 3 illustrates a closer view of the crucible 5 between posts 3, 4.

As illustrated in FIG. 4, the carbon crucible cavity 7 can then be loaded with radioactive Technetium from a prepared syringe 6. FIG. 5 illustrates a further close up of the loading process.

As illustrated in FIG. 6, subsequently, the draw is closed with the crucible in place (FIG. 6), and a pulsed current is passed between the two posts (3, 4 of FIG. 4). The pulse current causes flash heating of the crucible and Technetium (FIG. 7), so as to form a carbon encapsulated Technetium gas, which is subsequently passed out of the generator to the lungs of a patient for imaging (FIG. 8).

The process is then generally repeated, with the crucible 5 being consumed between iterations. The ends 9, 10 (CONTACTS) of posts 3, 4, are limited to about 50 iterations of crucibles and require replacement due to their overall deterioration.

### SUMMARY OF THE INVENTION

It is an object of the invention, in its preferred form to provide an improved form of crucible holder arrangement.

In accordance with a first aspect of the present invention, there is provided a post unit for holding one end of a carbon crucible utilised in the creation of a carbon encapsulated substance, the post unit including: a detachable contact unit including a first cavity for engaging one end of the carbon crucible; and at least a first and second pedestal for engaging with the contact unit; the first and second pedestals being resiliently clamped together around the first contact unit for engagement of the contact unit in a predetermined position.

In some embodiments the post unit further includes a lever for resiliently forcing the two pedestals apart for insertion and removal of the detachable contact unit. In some embodiments, the first contact unit includes a profiled surface which mates with a corresponding surface of at least one pedestal to engage the detachable contact unit in a predetermined orientation. In some embodiments, the profiled surface includes a ridge or slot or a knob or cavity.

### BRIEF DESCRIPTION OF THE DRAWINGS

Embodiments of the invention will now be described, by way of example only, with reference to the accompanying drawings in which:

FIG. 1 is an illustration of the Technegas generator of the prior art;

FIG. 2 illustrates opening the door of the Technegas generator;

FIG. 3 illustrates a close up of the crucible portions of FIG. 2;

FIG. 4 illustrates the process of loading the crucible with solute;

FIG. 5 illustrates a close up of the loading process;

FIG. 6 illustrates closing the door of the Technegas generator;

FIG. 7 illustrates schematically the process of heating of the crucible;

FIG. 8 illustrates schematically a patient breathing in the technetium products;

FIG. 9 illustrates an exploded perspective of the post structure of an embodiment;

FIG. 10 illustrates an alternative view of the exploded perspective of the post structure of an embodiment;

FIG. 11 and FIG. 12 illustrate exploded perspectives of a portion of the embodiment;

FIG. 13 illustrates the contact;

FIG. 14 illustrates the top pedestal;

FIG. 15 illustrates a first side perspective view of an alternative form of contact; and

FIG. 16 illustrates a second side perspective view of the alternative form of contact.

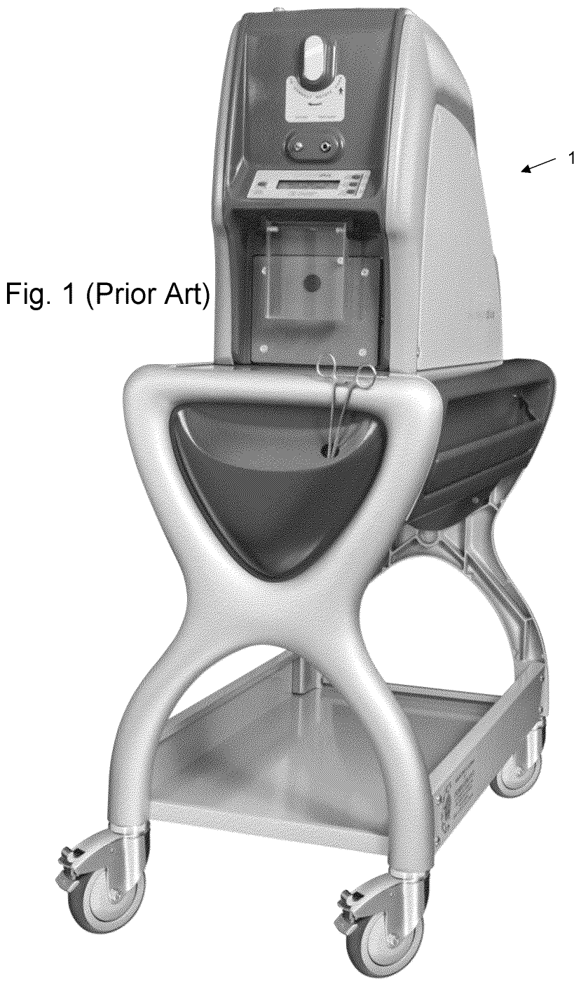

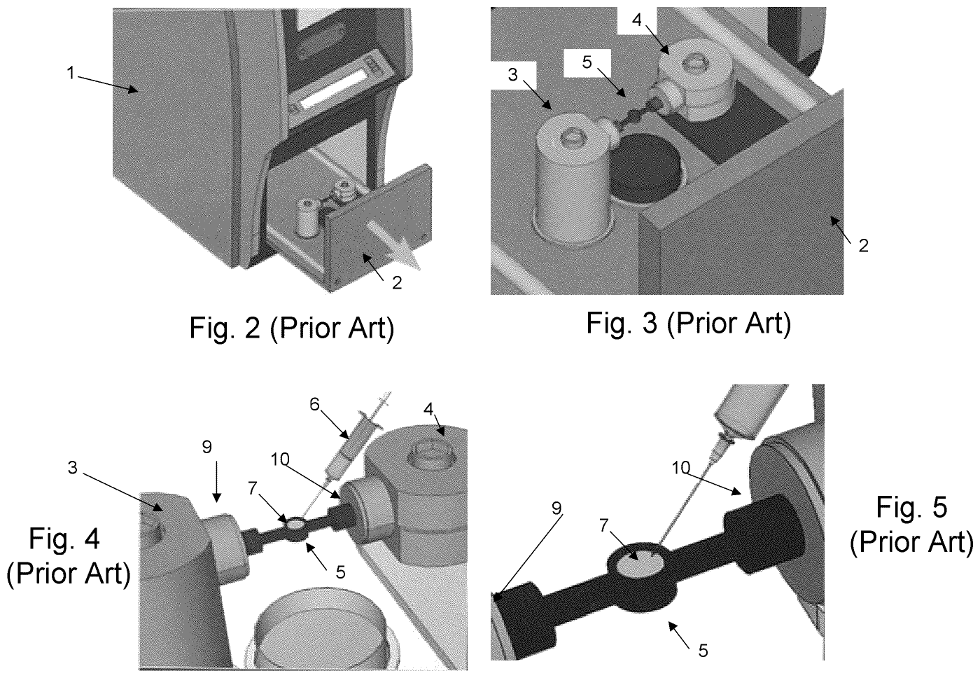

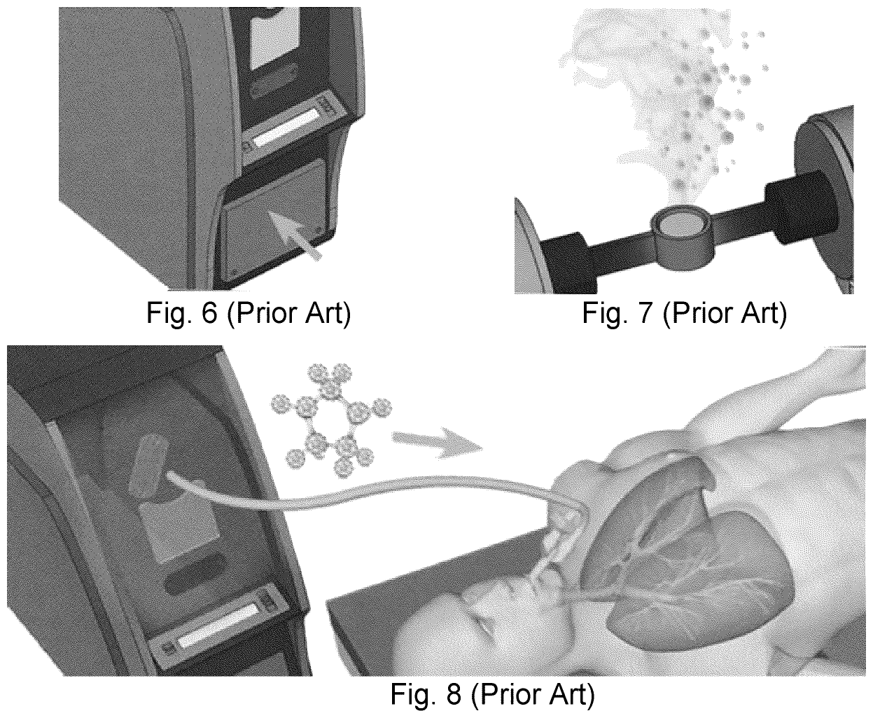

### DETAILED DESCRIPTION

The embodiments provide for a system and method which provides for an improved crucible holding arrangement, allowing for the simplified release of a replaceable crucible contact.

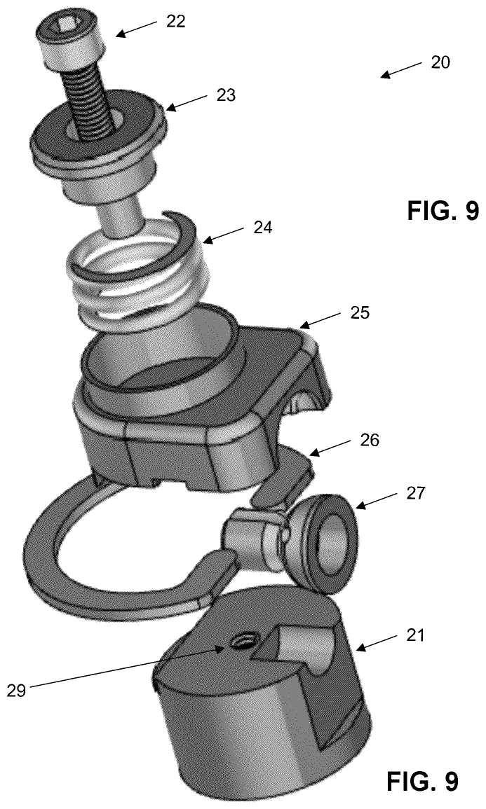

Turning initially to FIG. 9, there is illustrated a first exploded perspective alternative form of post 20 formed in accordance with an embodiment of the present invention.

The post 20 is designed to readily detach a conductive crucible contact 27 to which one end of the carbon crucible is attached.

The post 20 includes a bottom pedestal 21, a screw 22, a cap 23, a coil spring 24, a top pedestal 25, a lever 26 and the contact 27.

The screw 22 is engaged in aperture 29 of the bottom pedestal 21 so as to clamp the unit together around the contact 27.

Upon activation of the lever 26 in a downward direction, the two pedestals 25, 21 are resiliently forced apart under the resistance of spring 24, so as to allow for the removal and replacement of an old crucible contact 27 and the insertion of a new crucible contact. In this manner, the contact 27 can be simply and rapidly replaced. Upon release of the pressure on lever 26, the spring then brings the two pedestals back into firm contact around the crucible contact.

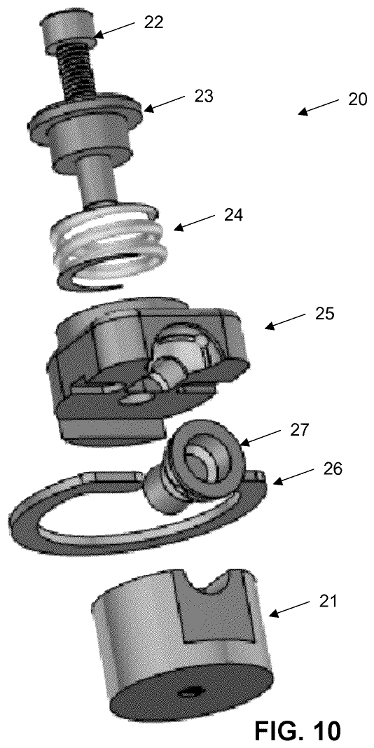

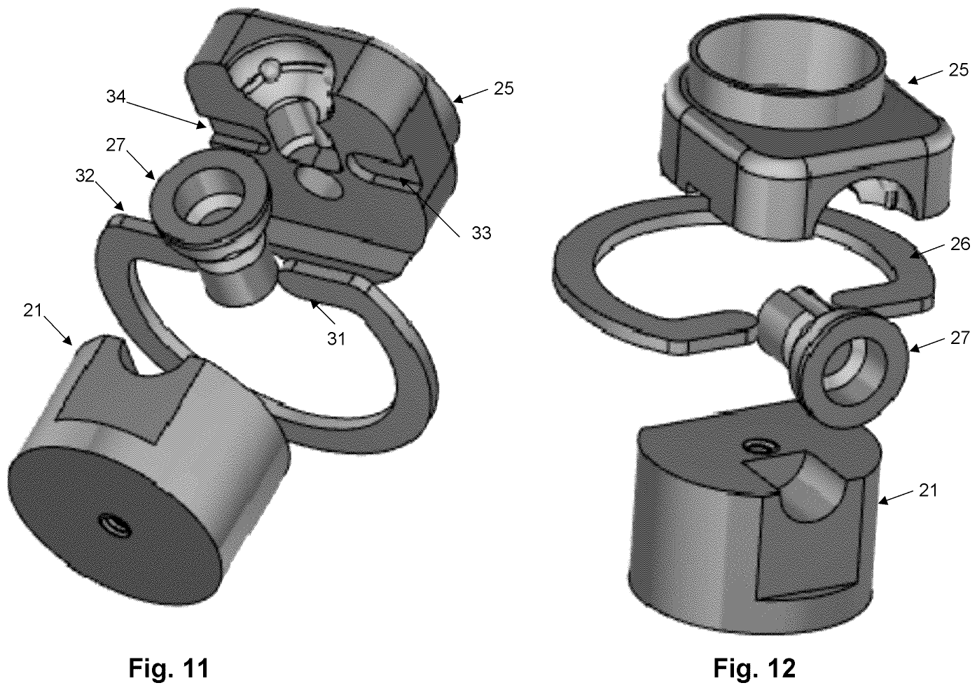

FIG. 10 illustrates a further exploded side perspective view of the post 20.

FIG. 11 illustrates a close-up perspective of the bottom pedestal 21, top pedestal 25, lever 26 and contact 27. The lever 26, includes tab ends 31, 32 which sit in recesses 33, 34 in normal operation. On activation of the lever 26, the ends act to force top pedestal 25 away from bottom pedestal 21.

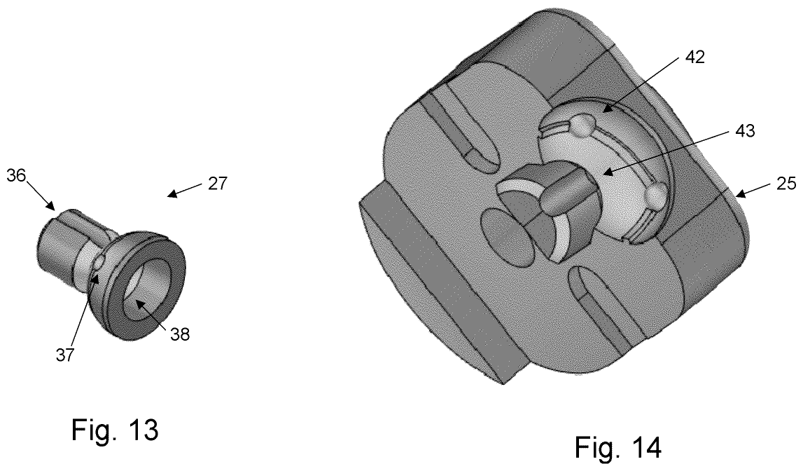

Turning briefly to FIG. 13, there is illustrated the crucible contact 27. The contact 27 includes a recess 38 for engaging the ends of a crucible. The contact 27 also includes a slot 36 and series of knobs e.g. 37. Turning to FIG. 14 there is illustrated the top pedestal 25. The top pedestal is designed to include ridge 43 and cavity 42 which engage with the slots and knobs of the contact to align the contact in place.

The embodiment therefore provides for an improved post arrangement whereby the contact can be rapidly and simply removed. The contact is firmly engaged in the post during normal operation and conductively connects to the post. The carbon crucible can then be mounted between two crucible contacts on opposed posts. The posts themselves can be retrofitted into the original generator.

Other embodiments of the invention are possible, including alternative placement of mating surfaces on the contact and pedestals.

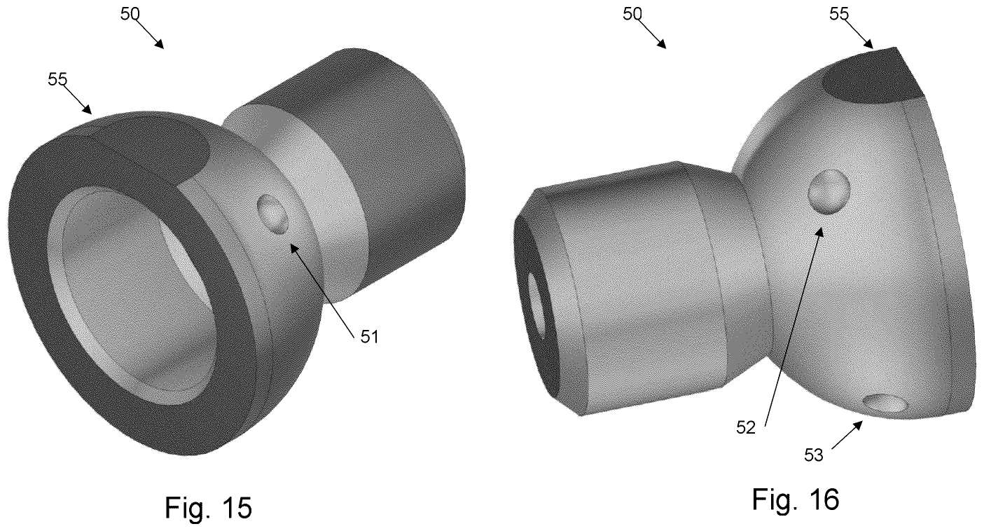

For example, turning now to FIG. 15 and FIG. 16 there is illustrates side perspective views of an alternative form of contact 50. This contact 50 is modified in a different manner than the earlier embodiment, to include a slot or chamfer 55 which orients the contact 50 upon insertion into a corresponding pedestal. Additionally, a series of shallow holes e.g. 51-53 are placed around the contact. The holes mate with corresponding bumps on insertion into the pedestal to firmly engage the contact in the pedestal. Alternatively, it will be evident that other arrangements of slots and holes can be provided as required.

The embodiments provide a new form of post arrangement which allows for the rapid in-situ replacement of the crucible contact which allows for rapid replenishment of the contact on demand.

### Interpretation

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A post unit for holding one end of a carbon crucible utilised in the creation of a carbon encapsulated substance, the post unit including:
a detachable contact unit including a first cavity for engaging one end of the carbon crucible; and
at least a first and second pedestal for engaging with the contact unit; the first and second pedestals being resiliently clamped together around the first contact unit for engagement of the contact unit in a predetermined position.

2. A post unit as claimed in claim 1, wherein said post unit is utilized in a technetium generator for the delivery of current and the mechanical holding of a carbon crucible via the post unit.

3. A post unit as claimed in claim 1 further comprising a lever for resiliently forcing the two pedestals apart for insertion and removal of the detachable contact unit.

4. A post unit as claimed in claim 1, wherein said detachable contact unit includes a profiled surface which maters with a corresponding surface of at least one pedestal to engage the detachable contact unit in a predetermined orientation.

5. A post unit as claimed in claim 4, wherein said profiled surface includes a ridge or slot or a knob or cavity.

6. A post unit as claimed in claim 3, wherein said lever includes a first profiled surface which engages with a corresponding slotted mating surface in at least one of said pedestals.

7. A detachable contact unit including a first cavity for engaging one end of a carbon crucible at a first proximal end, and a clamping surface located at a distal end for being detachably clamped to a conductive post.

8. A detachable contact unit as claimed in claim 7 further including a profiled surface at the distal end which includes at least one of a ridge, slot, knob or cavity.
